(19) United States
(12) Patent Application Publication
(10) Pub. No.: US 2024/0059725 A1
(43) Pub. Date: Feb. 22, 2024

(54) COUMARIN COMPOUNDS AND A PROCESS FOR PREPARATION THEREOF

(71) Applicant: Suman MAJUMDER, Kolkata (IN)

(72) Inventors: Suman MAJUMDER, Kolkata (IN); Atchuta Ramarao TEMMANABOYINA, Vishakapatnam (IN)

(73) Assignee: Suman MAJUMDER, Kolkata (IN)

(21) Appl. No.: 17/440,699

(22) PCT Filed: Aug. 19, 2021
(86) PCT No.: PCT/IB2021/057638

§ 371 Date: Nov. 17, 2022

(30) Foreign Application Priority Data
May 8, 2021 (IN) 202131020945

(51) Int. Cl. C07H 13/10; A61P 35/00; C07H 1/00

(52) U.S. Cl. CPC C07H 13/10; A61P 35/00; C07H 1/00

(57) ABSTRACT

The present invention relates to novel coumarin compound of formula (I) or a pharmaceutically acceptable salt thereof and pharmaceutical compositions containing them. The present invention also relates to a process for preparing the compound of formula (I) or a pharmaceutically acceptable salt thereof and use of the compound of formula (I) or a pharmaceutically acceptable salt thereof as a drug for their antioxidant and anticancer properties, and diagnostic purposes.

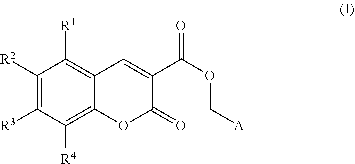

### FIELD OF THE INVENTION

The present invention generally relates to coumarin compounds. Specifically, the present invention relates to novel compound of formula (I) or a pharmaceutically acceptable salt thereof and pharmaceutical composition comprising the same. The present invention also relates to a process for preparing the compound of formula (I) or a pharmaceutically acceptable salt thereof and use of the compound of formula (I) or a pharmaceutically acceptable salt thereof as a drug and for diagnostic purposes.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

The coumarins are naturally occurring bio-active molecules and are known for their activity as pharmaceuticals, flavoring agents, fragrance agents, florescent probes and as anti-microbial and anti-viral agents.

Various studies have suggested that coumarins and their derivatives possess pharmacological properties such as anti-inflammatory, anti-coagulant, anti-bacterial, anti-fungal, anti-viral, anti-cancer, anti-hypertensive, anti-convulsant, anti-hyperglycemic, antioxidant and neuro-protective properties. One of the potential therapeutic activity for which coumarins are investigated is their anticancer activity.

Cancer remains a major health problem and considers to be the second most common cause of death worldwide. The rate of cancer-related mortality globally is at alarming level because of poor ability of prevention, diagnosis and efficient timely treatment of cancer. Besides, due to heterogeneity of cancer, there is still needed to develop effective anticancer drugs. Coumarins are characterized by a simple benzopyrone structure with multiple substitution sites and hence attracted attention of medicinal chemists to develop promising coumarin drug candidates for the treatment of cancer. However, coumarins are not hydrolysable and hence it is required to provide coumarin compounds which can be hydrolyzed by lysozymes at the blood brain barrier level and releases the drug like molecule from the prodrug.

Therefore, there remains an unmet need for new coumarin compounds for example those which are hydrolysable and can be a promising drug candidate effective against discase like cancer.

### OBJECTS OF THE INVENTION

An object of the present invention is to provide novel coumarin compounds and a process for preparation thereof.

Another object of the present invention is to provide coumarin compounds having improved hydrolysable characteristics and therapeutic potential for treating disease like cancer.

Another object of the present invention is to provide a process for preparation of a coumarin compounds or a pharmaceutically acceptable salt thereof that is simple, economical and results in high yield.

### SUMMARY OF THE INVENTION

The present invention generally relates to coumarins compounds. Specifically, the present invention relates to novel compound of formula (I) or a pharmaceutically acceptable salt thereof and pharmaceutical compositions containing them. The present invention also relates to a process for preparing the compound of formula (I) or a pharmaceutically acceptable salt thereof and use of the compound of formula (I) or a pharmaceutically acceptable salt thereof as a therapeutic or diagnostic purpose.

In one aspect, the present invention relates to a compound of formula (I) or a stereoisomer, a tautomer, a pharmaceutically acceptable salt or a pharmaceutically acceptable solvate thereof, in one aspect, the present invention relates to a compound of formula (I) or a stereoisomer, a tautomer, a pharmaceutically acceptable salt or a pharmaceutically acceptable solvate thereof,

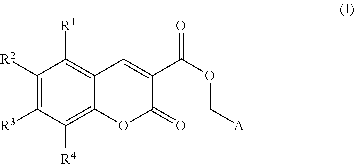

wherein:
R¹ to R⁴ are independently selected from the group consisting of unsubstituted or substituted (C₁₋₆) alkyl, unsubstituted or substituted (C₁₋₆) alkoxy, unsubstituted or substituted (C₃₋₆) cycloalkyl, form a (C4-10) heterocyclyl or (C6-10) aryl, halogen, and hydroxy; and
A represents ring A or ring A¹ as represented below,

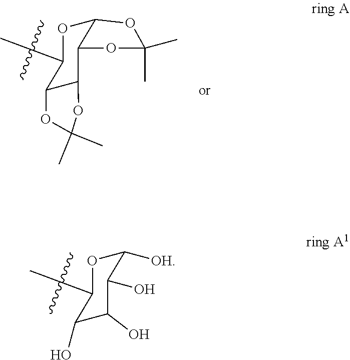 [SMILES: *C1OC2OC(C)(C)OC2C2OC(C)(C)OC12 ; *C1OC(O)C(O)C(O)C1O]

In another aspect, the present invention relates to a process for preparing a compound of formula (I) or a pharmaceutically acceptable salt thereof represented by a general scheme as shown below.

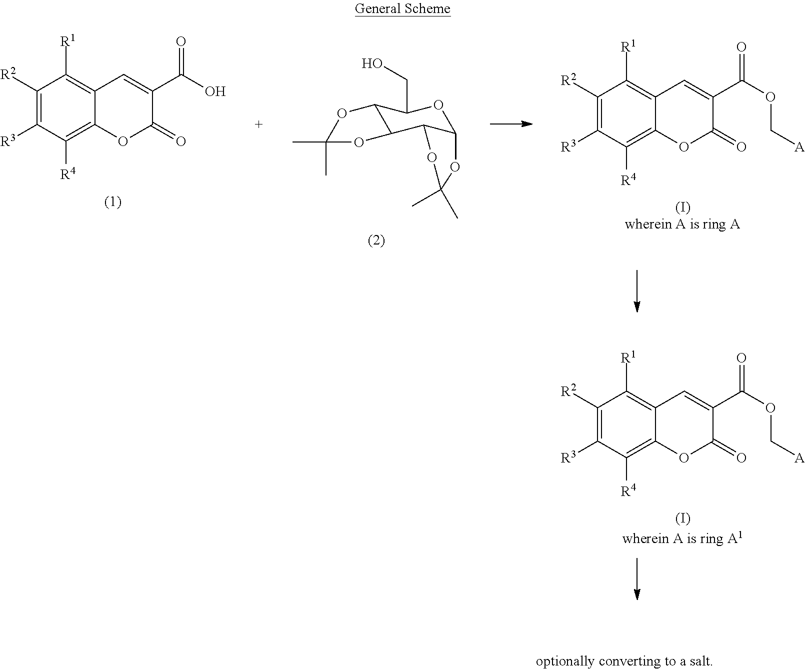

In one aspect, the present invention relates to a process for preparation of a compound of formula (I), or a pharmaceutically acceptable salt thereof comprising the steps of:
(a) reacting a compound of formula (1) with a compound (2) in presence of coupling agents and polar solvent(s) at a temperature in the range of 25° C. to 30° C. for a time period in the range of 6 hours to 48 hours to obtain a compound of formula (I) (with ring A);

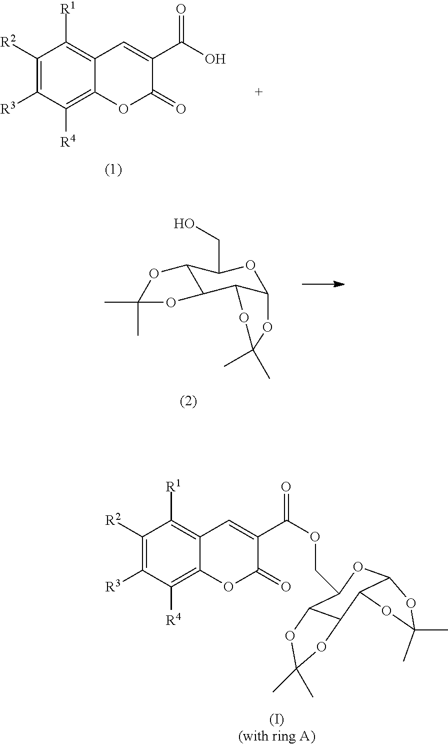

(b) reacting the compound of formula (Ia) (with ring A) obtained in step (a) with deprotecting agents in polar solvent(s) at a temperature in the range of 25° C. to 30° C. for a time period of 10 to 30 minutes to obtain a compound of formula (I) (with ring A¹); and

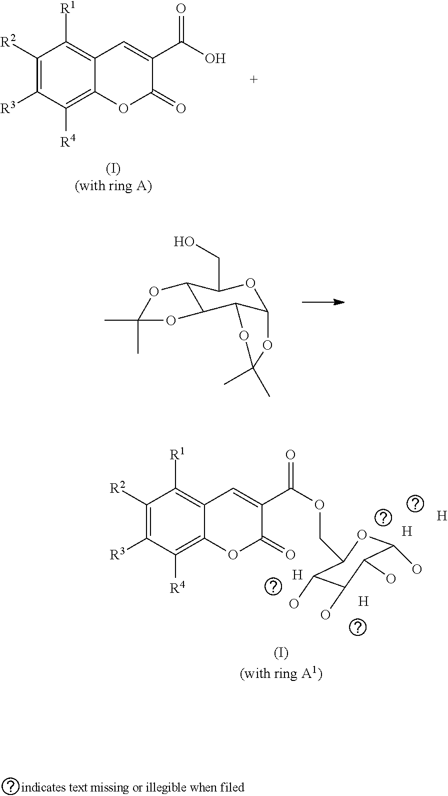

(C) optionally, converting the compound of formula (I) into its salt.

In yet another aspect, the present invention relates to a process for preparation of a compound (3) comprising the step of reacting compound (1a) with compound (2) in presence of coupling agents such as hexafluorophosphate azabenzotriazole tetramethyl uronium (HATU), tetrafluoroborate azabenzotriazole tetramethyl uronium (TATU), 2-(1H-Benzotriazole-1-yl)-1,1,3,3-tetramethylaminium tetrafluoroborate (TBTU), hexafluorophosphate benzotriazole tetramethyl uranium (HBTU), (1-Cyano-2-ethoxy-2-oxo ethylidenaminooxy)dimethylamino-morpholino-carbenium hexafluorophosphate (COMU) and the like, a base such as organic bases, N-methyl morpholine (NMM), imidazole, urea, carbamate and the like and polar solvent (s) to obtain the compound (3).

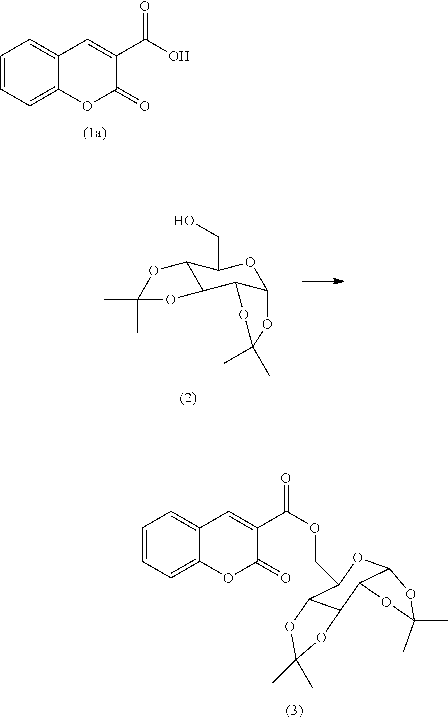

In yet another aspect, the present invention relates to a process for preparation of a compound (4) comprising the step of:
(a) reacting compound (1a) with compound (2) in presence of coupling agents such as HATU, TATU, TBTU, COMU and the like, a base such as organic bases, N-methyl morpholine (NMM), imidazole, urea, carbamate and the like and polar solvent (s) to obtain the compound (3); and
(b) deprotecting the compound (3) using deprotecting agents such as trifluoroacetic acid and the like and a polar solvent(s) to obtain a compound (4):

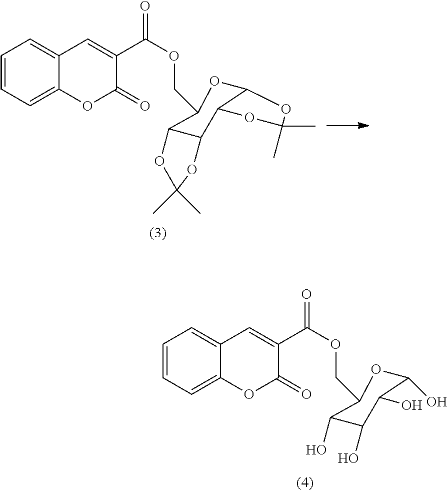 [SMILES: CC1(C)OC2OC(COC(=O)c3cc4ccccc4oc3=O)C3OC(C)(C)OC3C2O1 ; O=C(OCC1OC(O)C(O)C(O)C1O)c1cc2ccccc2oc1=O]

In another aspect, the present invention relates to a pharmaceutical composition comprising of a therapeutically effective amount of compound of formula (I) or a pharmaceutically acceptable salt thereof and pharmaceutically acceptable excipients.

In yet another aspect of the present invention, the pharmaceutical composition comprising of a compound of formula (I) is in the form of ready to use composition, powder, tablets and solution.

In yet another aspect, the present invention provides a cosmetic composition comprising of a compound of formula (I) or a pharmaceutically acceptable salt thereof.

In yet another aspect, the present invention provides a compound of formula (I) or a pharmaceutically acceptable salt thereof for use in the treatment of various diseases or disorders.

In yet another aspect, the present invention provides a compound of formula (I) or a pharmaceutically acceptable salt thereof for use as florescent probes to diagnose various diseases and disorders.

Various objects, features, aspects and advantages of the inventive subject matter will become more apparent from the following detailed description of preferred embodiments.

### BRIEF DESCRIPTION OF THE ACCOMPANYING DRAWING

Characteristics and advantages of the subject matter as disclosed in the present disclosure will become clearer from the detailed description of an embodiment thereof, with reference to the attached drawing, given purely by way of an example, in which:

FIG. 1 is NMR spectra of compound 3 as per one of the exemplary embodiments of the present invention.

FIG. 2 is NMR spectra of compound 4 as per one of the exemplary embodiments of the present invention.

FIG. 3 is bar graph depicting cytotoxicity data in the form of IC-50 value of the compound 4 in HEK 293 cell line.

FIG. 4 are graphs depicting Reactive Oxygen Species (ROS) production by compound 4 of the exemplary embodiments of the present invention in RAW cell line.

FIG. 5 is graph depicting activity of compound 3 of the exemplary embodiments of the present invention against Human colon carcinoma (HCT-116) cell line.

FIG. 6 is graph depicting activity of compound 6 of the exemplary embodiments of the present invention against Human breast adenocarcinoma (MDA-MB-468) cell line.

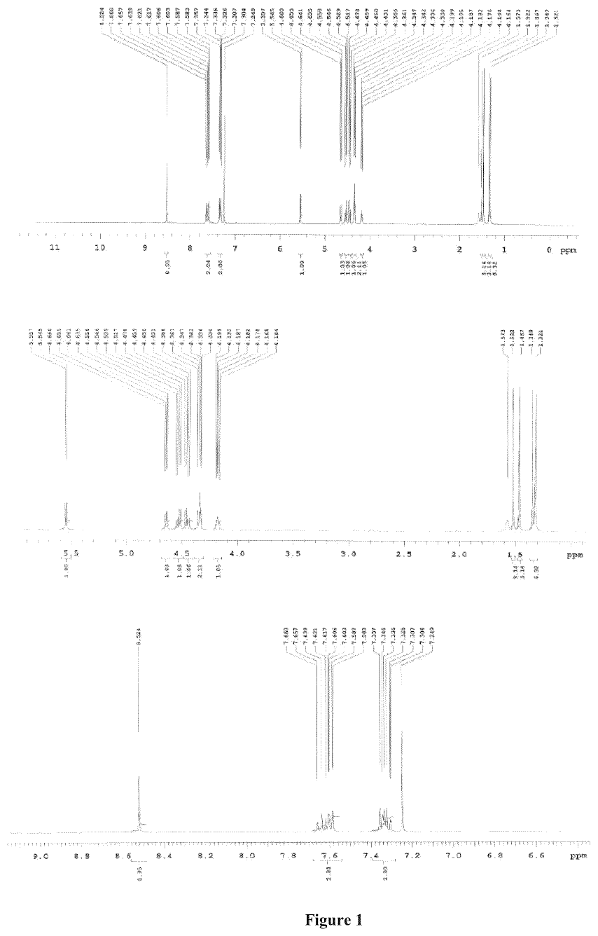

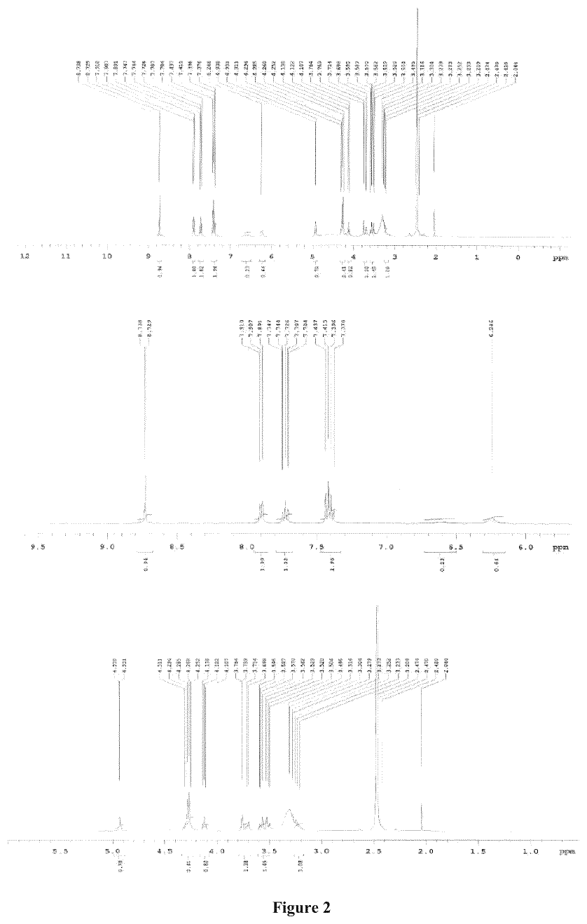

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if cach individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” or “another embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

Also, use of “(s)” as part of a term, includes reference to the term singly or in plurality, for example, the term pharmaceutically acceptable salt(s) indicates a single salt or more than one salt of the compound of formula (I).

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

The term, “or” as used herein, is generally employed in its sense including “and/or” unless the content clearly dictates otherwise.

The term, “(C1-6) alkyl”, as used herein, refers to the radical of saturated aliphatic groups, including straight or branched-chain alkyl groups having six or fewer carbon atoms in its backbone, for instance, C1-C6 for straight chain and C3-C6 for branched chain. As used herein, (C1-6) alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbuty.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term, “halogen” as used herein refers to chlorine, fluorine, bromine or iodine atom.

The term, “(C1-6) alkoxy” refers to a (C1-6) alkyl having an oxygen radical attached thereto. Representative examples of alkoxy groups include, but are not limited to, methoxy, ethoxy, propoxy, isopropoxy, n-butoxy, isobutoxy and tert-butoxy. Furthermore, unless stated otherwise, the alkoxy groups can be unsubstituted or substituted with one or more groups. A substituted alkoxy refers to a (C1-6) alkoxy substituted with one or more groups, particularly one to four groups independently selected from the groups indicated above as the substituents for the alkyl group.

The term “(C3-6) cycloalkyl” refers to a monocyclic hydrocarbon ring containing three to six carbon atoms. Representative (C3-6) cycloalkyl groups include, but are not limited to, cyclopropyl, cyclobutyl, cyclopentyl and cyclohexyl. Unless stated otherwise, (C3-6) cycloalkyl can be unsubstituted or substituted with one or more substituents, for example, substituents independently selected from the group consisting of oxo, halogen, (C1-6) alkyl, hydroxy, cyano, nitro and amine. Cycloalkyl group comprises a saturated cycloalkyl ring system which does not contain any double bond within the ring or a partially unsaturated cycloalkyl ring system which may contain one or more double bonds within the ring system that is stable, and do not form an aromatic ring system.

The term “(C6-10) aryl” or “aryl” as used herein refers to monocyclic or bicyclic hydrocarbon groups having 6 to 10 ring carbon atoms, wherein at least one carbocyclic ring is having a π electron system. Examples of (C6-C10) aryl ring systems include, but are not limited to, phenyl and naphthyl. Unless indicated otherwise, aryl group can be unsubstituted or substituted with one or more substituents, for example 1 -4 substituents independently selected from the group consisting of halogen, (C1-6) alkyl, hydroxy, cyano, nitro, —COOH and amino.

The term, “pharmaceutically acceptable salt(s)” as used herein includes a salt or salts of the active compound i.e. the compound of formula (I), which retain the desired biological activity of the subject compound and exhibit minimal undesired toxicological effects; and are prepared with suitable acids or bases, depending on the particular substituents found on the compounds described herein.

The term, “therapeutically effective amount” as used herein refers to an amount of a compound of formula (I) or a pharmaceutically acceptable salt thereof or a composition comprising a compound of formula (I) or a salt thereof, effective in producing the desired therapeutic response in a particular patient (subject) suffering from a disease or disorder.

The term “pharmaceutically acceptable excipient(s)” as used herein refers to a diluent, binder, disintegrant, glidant, lubricant, coating material or the like, which is non-toxic, and inert, which does not have undesirable effects on a subject to whom it is administered and is suitable for delivering a therapeutically active agent to the target site without affecting the therapeutic activity of the said agent.

The term, “subject” as used herein refers to an animal, preferably a mammal, and most preferably a human. The term “mammal” used herein refers to warm-blooded vertebrate animals of the class ‘mammalia’, including humans, characterized by a covering of hair on the skin and, in the female, milk-producing mammary glands for nourishing the young, the term mammal includes animals such as cat, dog, rabbit, bear, fox, wolf, monkey, deer, mouse, pig and human.

The terms, “treatment”, “treat” and “therapy” and the like as used herein refer to alleviate, slow the progression, attenuation, prophylaxis or as such treat the existing diseases or condition. Treatment also includes treating, preventing development of, or alleviating to some extent, one or more of the symptoms of the diseases or condition.

In an embodiment, the present invention relates to all the compounds described by a compound of formula (I) or a pharmaceutically acceptable salt thereof;

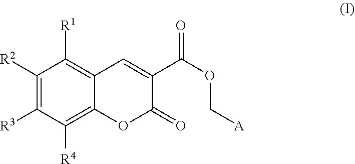

wherein:
R1 to R4 are independently selected from the group consisting of unsubstituted or substituted (C₁₋₆) alkyl, unsubstituted or substituted (C₁₋₆) alkoxy, unsubstituted or substituted (C₃₋₆) cycloalkyl, form a (C₄₋₁₀) heterocyclyl, (C₆₋₁₀) aryl, halogen, and hydroxy; and
A represents ring A or ring A¹ as represented below,

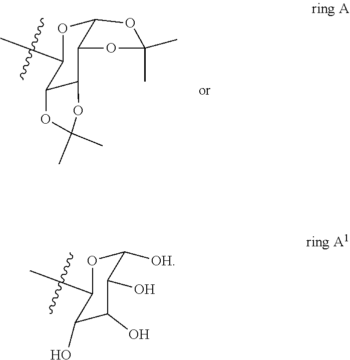 [SMILES: *C1OC2OC(C)(C)OC2C2OC(C)(C)OC12 ; *C1OC(O)C(O)C(O)C1O]

In a specific embodiment, the present disclosure provides a compound of formula (3):

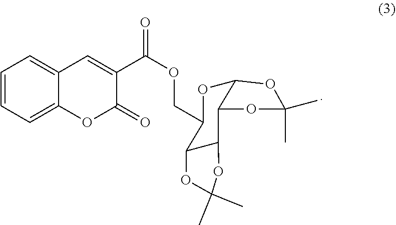 [SMILES: CC1(C)OC2OC(COC(=O)c3cc4ccccc4oc3=O)C3OC(C)(C)OC3C2O1]

In one specific embodiment the present disclosure provides a compound of formula (4):

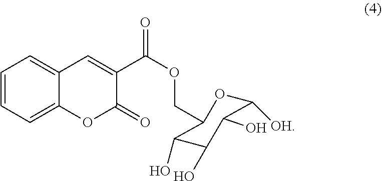 [SMILES: O=C(OCC1OC(O)C(O)C(O)C1O)c1cc2ccccc2oc1=O]

According to the present invention, the compound of formula (I) are more hydrolysable as compared to the conventional anticancer compound as can be seen from the experimental results, wherein the compound of formula (I) shows lower cytotoxicity against normal human cells and illustrating better flexibility in polarity and hydrolysable characteristics of the compound of formula (I).

According to the present invention, the compound of formula (I) comprising the glycol-ester bond to the active coumarin molecules can help the compound to have a lipolizable glucoester bond attached to the molecule. Thus, the compounds of the present invention have highly potential to show anti-oxidant property, and anti-cancer activity.

In another embodiment, the present disclosure provides to a process for preparing a compound of formula (I) or a pharmaceutically acceptable salt thereof represented by a general scheme as shown below.

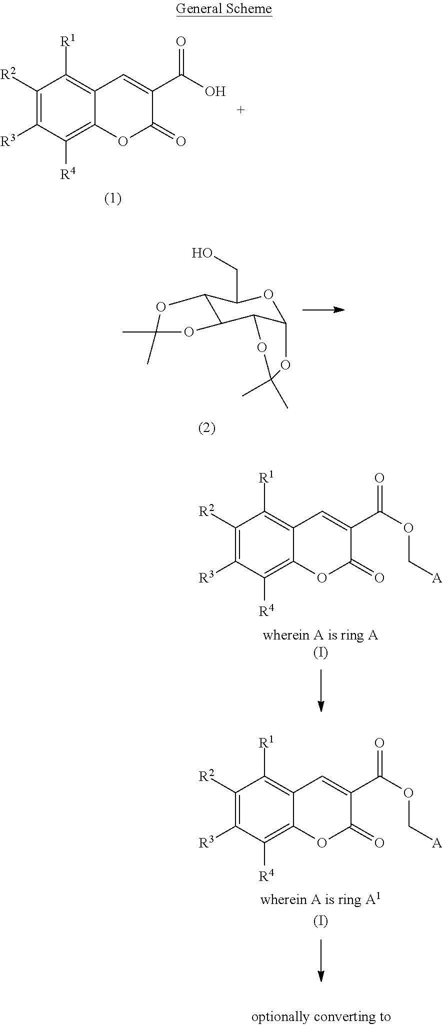

a salt.

In one embodiment, the present disclosure provides a process for preparation of a compound of formula (I), or a pharmaceutically acceptable salt thereof comprising the steps of:
(a) reacting a compound of formula (I) with a compound (2) in presence of coupling agents and polar solvent(s) at a temperature in the range of 25° C. to 30° C. for a time period in the range of 6 hours to 48 hours to obtain a compound of formula (I) (with ring A);

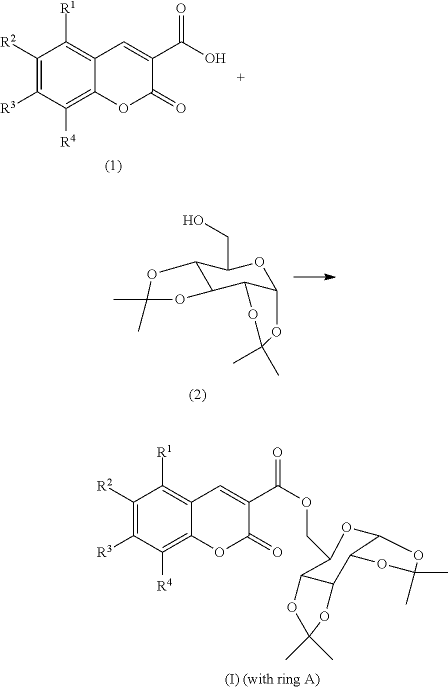

(b) reacting the compound of formula (I) (with ring A) obtained in step (a) with deprotecting agents in polar solvent(s) at a temperature in the range of 25° C. to 30° C. for a time period of 10 to 30 minutes to obtain a compound of formula (I) (with ring A¹); and

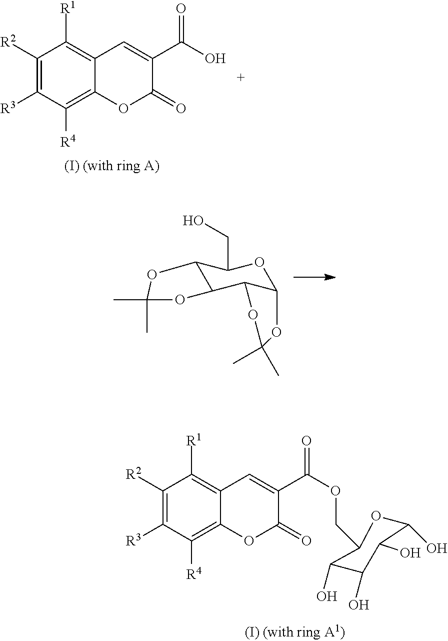

(C) optionally, converting the compound of formula (I) into its salt.

In yet another embodiment, the present disclosure provides a process for preparation of a compound (3) comprising the step of reacting compound (1a) with compound (2) in presence of coupling agent(s), a base and polar solvent(s) to obtain the compound (3).

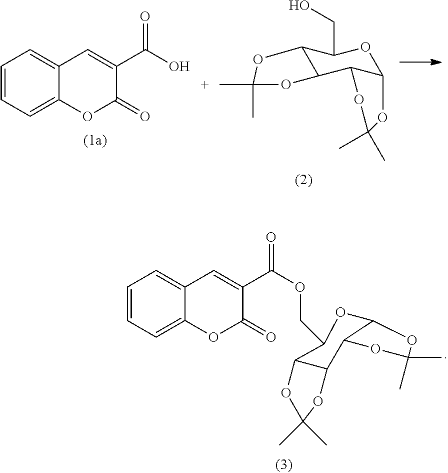

In still another embodiment, the present disclosure provides a process for preparation of a compound (4) comprising the step of:
(a) reacting compound (1a) with compound (2) in presence of coupling agent(s), a base and polar solvent(s) to obtain the compound (3); and

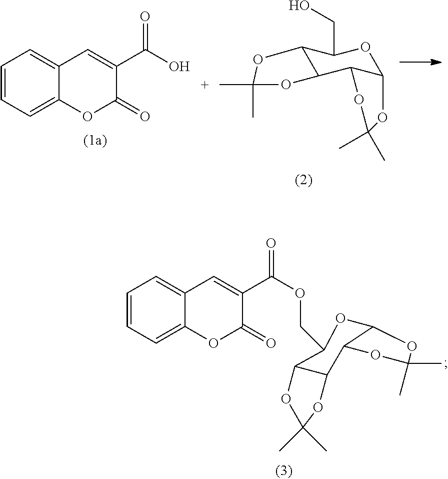

and
(b) deprotecting the compound (3) using deprotecting agent(s) and polar solvent(s) to obtain a compound (4):

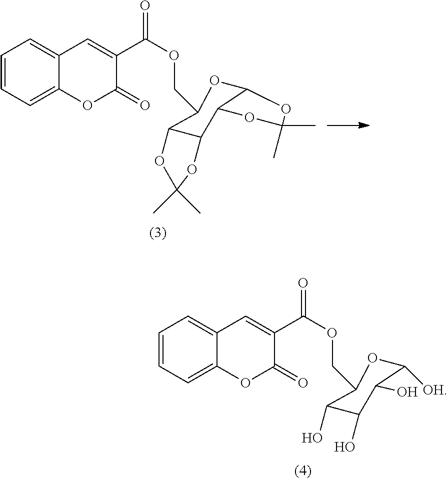 [SMILES: CC1(C)OC2OC(COC(=O)c3cc4ccccc4oc3=O)C3OC(C)(C)OC3C2O1 ; O=C(OCC1OC(O)C(O)C(O)C1O)c1cc2ccccc2oc1=O]

In one embodiment the present disclosure provides a process for preparing a compound (3) or compound (4), the process comprising the step of: (a) reacting compound (1a) with compound (2) in presence of coupling agent(s), a base and polar solvent(s) at a temperature in the range of 25° C. to 30° C. for a time period in the range of 6 hours to 48 hours to obtain the compound (3); and

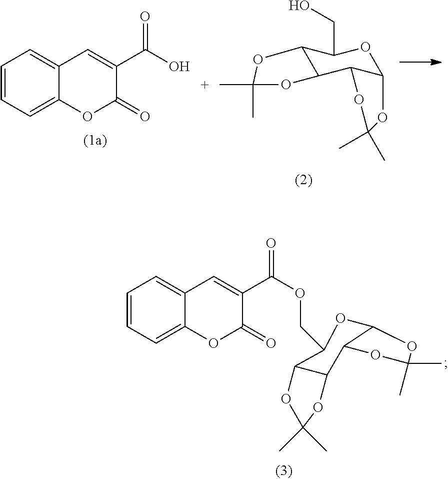

optionally
(b) deprotecting the compound (3) using deprotecting agent(s) and polar solvent(s) at a temperature in the range of 25° C. to 30° C. for a time period of 10 to 60 minutes to obtain a compound (4):

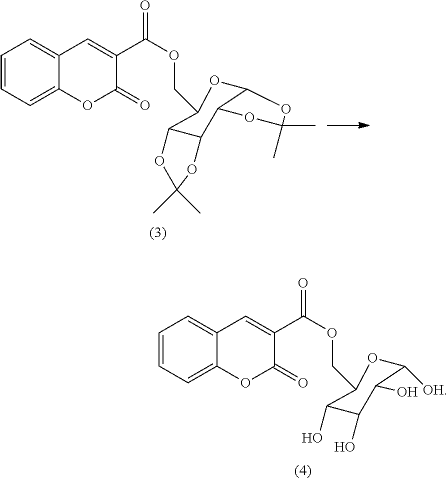 [SMILES: CC1(C)OC2OC(COC(=O)c3cc4ccccc4oc3=O)C3OC(C)(C)OC3C2O1 ; O=C(OCC1OC(O)C(O)C(O)C1O)c1cc2ccccc2oc1=O]

In one embodiment the coupling agent(s) can be selected from the group consisting of but not limited to hexafluorophosphate azabenzotriazole tetramethyl uronium (HATU), tetrafluoroborate azabenzotriazole tetramethyl uronium (TATU), 2-(1H-Benzotriazole-1-yl)-1,1,3,3-tetramethylaminium tetrafluoroborate (TBTU), hexafluorophosphate benzotriazole tetramethyl uranium (HBTU), (1-Cyano-2-ethoxy-2-oxo ethylidenaminooxy)dimethylamino-morpholino-carbenium hexafluorophosphate (COMU), or the mixture thereof.

In one embodiment the base can be an organic bases selected from the group consisting of but not limited to N-methyl morpholine (NMM), imidazole, urea, carbamate, pyridine, N,N-diisopropylamine and triethylamine and the like or mixture thereof.

In an embodiment the polar solvent can be selected from but not limited to water, dichloromethane, tetrahydrofuran, ethyl acetate, dimethylformamide, acetonitrile, dimethyl sulfoxide, alcohol or a mixture thereof.

In an embodiment the deprotecting agent(s) can be selected from the group consisting of but not limited to trifluoroacetic acid, aqueous tert-butyl hydroperoxide (70%), para toluene sulphonic acid, zirconium chloride, and indium trichloride.

According to the present invention, the coupling reaction of the present process using carboxylic acids (here coumarins-3-carboxylic acid and its compounds) and the acetonide protected glucose using various coupling agents is unique. The advantage is that it is a direct coupling with the acid group and the protected glucose instead of derivatizing the acid to either an acid chloride (in situ) or benzotriozolyl derivative. Thus, the process according to the present disclosure is an energy efficient process and provides high yield unlike the other known methods.

In another embodiment, the compound of formula (I) can be converted into a pharmaceutically acceptable salt. The pharmaceutical acceptable salts of the compound of formula (I) according to the invention are prepared in a manner known to one skilled in the art. Pharmaceutically acceptable salts of the compound of the present invention include but not limited to, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

In yet another embodiment, the pharmaceutically acceptable salts of the compound of the present invention containing acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

In another embodiment, the present disclosure provides a pharmaceutical composition comprising a therapeutically effective amount of a compound of formula (I) or its pharmaceutically acceptable salt and pharmaceutically acceptable excipients.

The present invention also relates to a process for the preparing pharmaceutical composition comprising a therapeutically effective amount of a compound of formula (I) or its pharmaceutically acceptable salt, which includes bringing a compound of formula (I) or salt thereof, into a suitable administration form using a pharmaceutically acceptable excipient or a carrier and, if appropriate, further suitable pharmaceutically acceptable carriers, filler, additives or auxiliaries. The pharmaceutical compositions containing the compound of formula (I) according to the invention are prepared in a manner known to one skilled in the art.

In certain embodiments, the pharmaceutical composition comprising the compound of formula (I) can be formulated into a variety of dosage forms such as tablets, delayed release or sustained release tablets powders, lozenges, pills, capsules, granules, syrups, elixirs, injectable solutions or suspensions, ointments, creams, transdermal patches, suppositories, aerosols or nasal sprays and the like. These pharmaceutical compositions can, if desired, contain additional ingredients such as flavorings, binders, excipients and the like.

In certain embodiment the pharmaceutical composition comprising the compound of formula (I) can be formulated to treat the underlying etiology with an effective amount administering the patient in need by oral administration, delayed release or sustained release, transmucosal, syrup, topical, parenteral administration, injection, subdermal, oral solution, rectal administration, buccal administration or transdermal administration.

In an embodiment, the pharmaceutical compositions can be administered orally, for example in the form of pills, tablets, coated tablets, capsules, granules or elixirs. Administration, however, can also be carried out rectally, for example in the form of suppositories; or parenterally, for example, intravenously, intramuscularly or subcutaneously, in the form of injectable sterile solutions or suspensions; or topically including buccal and sublingual, rectal, vaginal, for example in the form of ointments or creams or transdermally, in the form of patches, films, or in other ways, for example in the form of aerosols or nasal sprays.

For the production of oral dosages form of the compound of formula (I) such as the pills, tablets, coated tablets, delayed release or sustained release tablets and hard gelatin capsules, it is possible to use, for example, lactose, corn starch or compounds thereof, gum arabica, magnesia or glucose, etc. Pharmaceutically acceptable excipients that can be used for soft gelatin capsules and suppositories are, for example, fats, waxes, natural or hardened oils, etc. Suitable pharmaceutically acceptable excipients for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the said solvents.

Methods of preparing various pharmaceutical compositions with a certain amount of compound of formula I, optionally in combination with other active agents are known, or will be apparent in light of this disclosure, to those skilled in this art from the disclosure in various known publications and literatures relevant to the art for example Remington's Pharmaceutical Sciences, Mack Publishing Company, Easton, Pa., 19th Edition (1995).

In certain embodiments, the pharmaceutical compositions described herein are formulated in a manner such that said compositions will be delivered to a patient in a therapeutically effective amount, as part of a prophylactic or therapeutic treatment. The desired amount of the composition to be administered to a patient will depend on absorption, inactivation, and excretion rates of the drug as well as the delivery rate of the drug (compound of formula (I)) and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

In certain embodiment, the pharmaceutical compositions described herein will incorporate the disclosed compound of formula (I) or salt thereof a drug to be delivered in an amount sufficient to deliver to a patient a therapeutically effective amount of a compound of formula (I) or salt thereof as part of a prophylactic or therapeutic treatment. The desired concentration of compound of formula (I) or salt thereof will depend on absorption, inactivation, and excretion rates of the drug as well as the delivery rate of the drug from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of the compound or salt may be adjusted to accommodate variations in the treatment parameters. Such treatment parameters include the clinical use to which the preparation is put, e.g., the site treated, the type of patient, e.g., human or non-human, adult or child, and the nature of the disease or condition.

The concentration and/or amount of compound of formula (I) or salt thereof may be readily identified by routine screening in animals, e.g., rats, by screening a range of concentration and/or amounts of the material in question using appropriate assays. Known methods are also available to assay local tissue concentrations, diffusion rates of the compositions, and local blood flow before and after administration of therapeutic formulations disclosed herein. One such method is microdialysis (reviewed by T. E. Robinson et al., 1991, microdialysis in the neurosciences, Techniques, volume 7, Chapter 1). The method reviewed by Robinson may be applied, in brief, as follows. A microdialysis loop is placed in situ in a test animal. Dialysis fluid is pumped through the loop. When compound of formula (I) is injected adjacent to the loop, released drug is collected in the dialysate in proportion to their local tissue concentrations.

In certain embodiments, the dosage of the subject compositions provided herein may be determined by reference to the plasma concentrations of the therapeutic composition or other encapsulated materials. For example, the maximum plasma concentration (Cmax) and the area under the plasma concentration-time curve from time 0 to infinity may be used.

Generally, in carrying out the methods detailed in this application, an effective dosage for the compound of formula (I) is in the range of about 0.01 mg/kg/day to about 100 mg/kg/day in single or divided doses, for instance 0.01 mg/kg/day to about 50 mg/kg/day in single or divided doses. The compound of formula (I) may be administered at a dose of, for example, less than 0.2 mg/kg/day, 0.5 mg/kg/day, 1.0 mg/kg/day, 5 mg/kg/day, 10 mg/kg/day, 20 mg/kg/day, 30 mg/kg/day, or 40 mg/kg/day. Compound of formula (I) may also be administered to a human patient at a dose of, for example, between 0.1 mg and 1000 mg, between 5 mg and 80 mg, or less than 1.0, 9.0, 12.0, 20.0, 50.0, 75.0, 100, 300, 400, 500, 800, 1000, 2000, 5000 mg per day.

Illustratively, the amount of the compound of formula (I) or its pharmaceutically acceptable salt in the pharmaceutical compositions normally is from about 1 to 500 mg or may be lower than or higher than the lower and the upper limit respectively. The dose of the compound of formula (I), which is to be administered, can cover a wide range depending on the type of disease or disorder to be treated. The dose to be administered daily is to be selected to suit the desired effect. A suitable dosage is about 0.01 to 100 mg/kg of the compound of formula (I) or its pharmaceutically acceptable salt depending on the body weight of the recipient (subject) per day, for example, about 0.1 to 50 mg/kg/day of a compound of formula (I) or a pharmaceutically acceptable salt of the compound. If required, higher or lower daily doses can also be administered.

In another embodiment, the pharmaceutical compositions normally contain about 0.01% to 99%, for example, about 1% to about 70%, about 5% to about 50%, or from about 10% to about 30% by weight of the compound of formula (I) or its pharmaceutically acceptable salt and for parenteral use in a concentration of from about 0.05 to about 50% w/v of the composition and preferably from about 5 to about 20% w/v of the compound of formula (I) or its pharmaceutically acceptable salt.

The selected dosage level will depend upon a variety of factors including the activity of a compound of the present invention, or its salt employed, the route of administration, the time of administration, the rate of excretion of the particular compound being administered, the duration of the treatment, other concurrently administered drugs, compounds and/or materials, the age, sex, weight, condition, general health and prior medical history of the patient (subject) being treated, and like factors well known in the medical arts.

In addition to the compound of formula (I) or its pharmaceutically acceptable salt and the pharmaceutically acceptable carrier substances, the pharmaceutical compositions of the present invention can contain additives such as, for example, fillers, antioxidants, dispersants, emulsifiers, defoamers, flavors, preservatives, solubilizers or colorants. Furthermore, in addition to a compound of formula (I) or its pharmaceutically acceptable salt, the pharmaceutical compositions can also contain one or more other therapeutically or prophylactically active agents.

The compounds of the present invention have shown high anti-cancer activity and accordingly are effective drug candidate for the treatment of cancers.

The present invention also encompasses within its scope the use of a compound of formula (I) or its pharmaceutically acceptable salt in combination, with other therapeutically active agents.

In an embodiment, the combination of compound of present invention with another therapeutic agent or treatment includes co-administration of a compound of formula (I) with the other therapeutic agent or treatment as either a single combination dosage form or as multiple, separate dosage forms, administration of the compound of the present invention first, followed by the other therapeutic agent or treatment and administration of the other therapeutic agent or treatment first, followed by the compound of present invention. Further therapeutic agents are administered either simultaneously or sequentially.

In another embodiment of the present invention, the other therapeutic agent may be any agent that is known in the art to treat, prevent, or reduce the symptoms of a disease or disorder. The selection of other therapeutic agent(s) is based upon the particular disease or disorder being treated. Such choice is within the knowledge of a treating physician. Furthermore, the additional therapeutic agent may be any agent when administered in combination with the administration of a compound of the present invention provides benefit to the subject in need thereof.

In another embodiment, the present invention relates to a method of treatment of anti-inflammatory diseases or disorders, neurodegenerative diseases or disorders, and cancer diseases comprising administering a therapeutically effective amount of the compound of formula (I) or a pharmaceutically acceptable salt thereof to a subject in need thereof.

In another embodiment, the compounds of the present invention are florescent tagged marker that can be used as inhibitors as well as florescent measurement through change in florescent emission in in-vitro assays.

In yet another embodiment, the compounds of the present invention are florescent tagged marker for radio-diagnostic use in cancer patients.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

### Preparation of Compound (3)

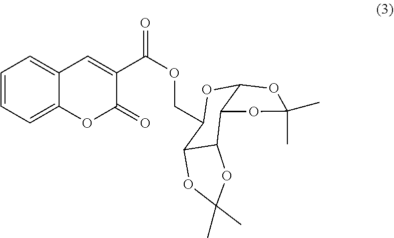 [SMILES: CC1(C)OC2OC(COC(=O)c3cc4ccccc4oc3=O)C3OC(C)(C)OC3C2O1]

To a solution of coumarin-3-carboxylic acid (5.0 g, 26.2 mmol, 1.0 eq) in dry DMF (30 mL) was added HATU (11.99 g, 31.5 mmol, 1.2eq) under nitrogen atmosphere. N-methylmorpholine (5.31 g, 52.5 mmol, 2eq) was added drop wise and the reaction was stirred for 5 minutes at room temperature (RT). The diisopropylidene galactose (compound (2), 8.21 g, 31.5 mmol, 1.2 eq) was dissolved in DMF (10 mL) and added drop wise to the above mixture under N₂-atm. The progress of the reaction was monitored by TLC (Mobile phase: 1:1 Ethyl acetate/Hexane), observed complete conversion. The reaction mixture was added drop wise to a mixture of 1:1 water and brine (300 mL) solution and stirred for 30 minutes. The precipitated product was filtered, washed successively with water (50 mL), saturated bicarbonate solution (50 mL), diethyl ether (50 mL), concentrated under vacuum and dried to obtain the compound of formula (3).

Yield: 11 g (97%); Purity: 99.3%; LCMS: m/z 433.42 [(M+H)]⁺.

¹H NMR (400 Mhz, CDCl₃): δ 8.52 (s, 1H), 7.66-7.58 (m, 2H), 7.35-7.30 (m, 2H), 5.55 (d, 1H), 4.64 (dd, IH), 4.55-4.51 (m, IH), 4.47-4.43 (m, 1H), 4.36-4.33 (m, 2H), 4.19-4.16 (m, 1H), 1.52 (s, 3H), 1.46 (s, 3H), 1.34 (s, 3H), 1.32 (s, 3H).

### Example 2

### Preparation of Compound (4)

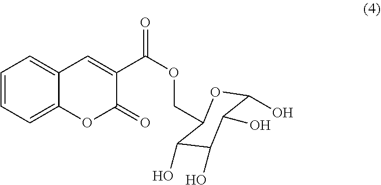 [SMILES: O=C(OCC1OC(O)C(O)C(O)C1O)c1cc2ccccc2oc1=O]

The compound (3) obtained in Example 1 was charged (5 g, 11.5 mmol, 1 eq) into a 2 neck RB, and a mixture of TFA (40 mL, 8 vol) and water (10 mL, 2 vol) was charged slowly over a period of 10 minutes. The reaction mixture was stirred for 20-30 minutes, progress of the reaction was monitored by TLC (Mobile phase: 1:1 Ethyl acetate/Hexane), observed complete conversion. The reaction mass was transferred to a single neck RB and concentrated in rotavapor at 40° C. under vacuum to remove excess TFA. To the residue was added acetonitrile (50 mL) and stirred for 30 minutes. Filtered and washed the solid product with diethyl ether (50 mL), suck dried under vacuum for one hour and dried to obtain the compound (4).

Yield: 3.9 g (96%); Purity: 96.6; LCMS: m/z 375.28 [(M+Na)]⁺. ¹H NMR (400 Mhz, CDCl₃): δ 8.72 (s, 1H), 7.90 (dd, 1H), 7.72 (dt, 1H), 7.43-7.37 (m, 2H), 6.24 (br s, 1H), 4.93 (s, 1H), 4.31-4.25 (m, 2H), 4.12 (dd, 1H), 3.75 (s, IH), 3.59-3.50 (q, 1H), 3.31-3.23 (m, 1H)

### Example 3

### Cytotoxicity Assay Report for DP (Compound 4)

### Cell Culture

Human embryonic kidney HEK 293 cell line were cultured in complete DMEM (Dulbecco's modified eagle medium) high glucose medium with 10% foetal bovine serum and 1% Penicillin Streptomycin and grown at 37° C.in a humidified incubator with 5% CO2. The cells were grown in T-25 flasks till about 80% confluency and 5000cells/well were seeded in a 96 well cell culture plate.

### Drug Treatment

After 24 hr the cells were treated with all the concentrations of the drug (compound (4)) in triplicates and incubated for 48 hr.

### MTT Assay

After 48 hr of incubation MTT was added at a concentration of 0.5 mg/ml and incubated at 37° C. for 4 hr. After 4 hr the media was discarded from each well and DMSO was added. After 30 mins of incubation at 37° C.the colorimetric data was taken at 570 nm.

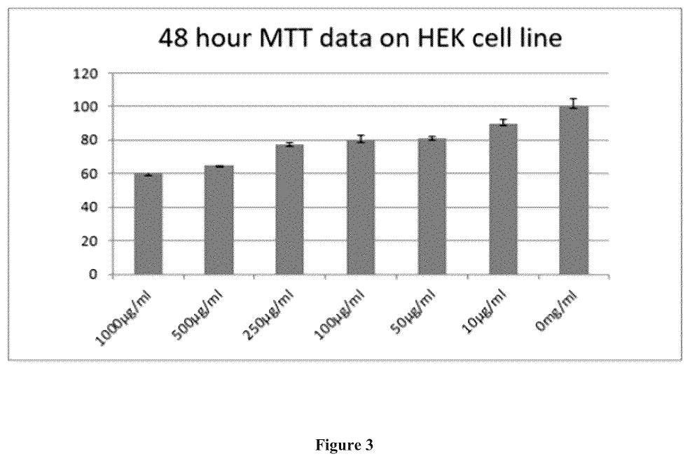

As can be seen from FIG. 3 the cytotoxicity data shows that the IC-50 value of the drug is greater than 1 mg/ml in HEK 293 cell line. Data showing the effect of the drug on HEK 293 cell line after 48 hr of treatment.

### Example 4

### Effect of Compound (4) on ROS Production in RAW 264.7 Cells and Radical Scavenging Activity

### Cell Culture

RAW cell line was cultured in a T 25 flask in DMEM. After the cells reached confluency evel up to 90 percent, they were seeded in 6 well plates at 1 lakh cells/well. After adhesion of cells drugs were treated along with LPS and kept for 48 hours.

Flow cytometry analysis of Reactive Oxygen Species production on RAW cell line:

After 48 hr of treatment cells were harvested and stained with DCFDA and run-on flow cytometer.

### Results

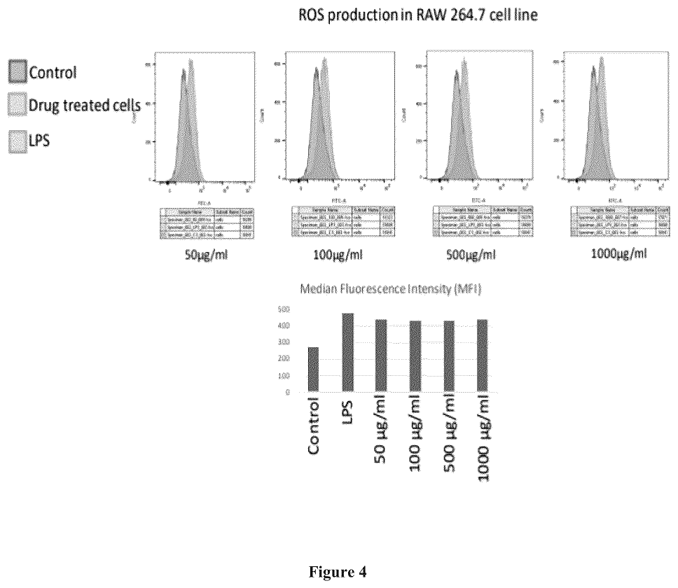

As can be seen from FIG. 4 flowcytometric analysis of ROS production showed the compound were able to produce ROS in all concentrations in RAW cell line that is not concentration dependent. The ability of compound (I) in reduction of intracellular ROS production, through generation of ROS in LPS induced RAW cell line, which are able to quench the inflammation triggered ROS in the cell, relates to strong anti-oxidant property of the molecule. However, no ROS production were observed in HEK cell line.

### Example 5

Anticancer activity of Compound (3) against Human colon carcinoma (HCT-116) cell lines and Compound (4) against Human breast adenocarcinoma (MDA-MB-468)

The anticancer activity of the compound (3) denoted as “P” and compound (4) denoted as “DP” was assessed invitro by MTT 3-(4,5-dimethylthiazol-2-yl)-2,5-diphenyltetrazolium bromide assay against Human colon carcinoma (HCT-116) cell lines and Human breast adenocarcinoma (MDA-MB-468) respectively.

RPMI-1640, fetal bovine serum (FBS), penicillin, streptomycin was procured from HiMedia (Mumbai, India). Doxorubicin, MTT 3-(4,5-dimethylthiazol-2-yl)-2,5-diphenyltetrazolium bromide were procured from Sigma Aldrich (USA). The chemicals used were of analytical or HPLC grade. Human breast adenocarcinoma (MDA-MB-468), Human colon carcinoma (HCT-116) was procured from NCCS, Pune, India. The cell line was grown in the RPMI-1640 with 2 mM L-glutamine, supplemented with 10% FBS, penicillin (100 IU/mL) and streptomycin (100 μg/mL) at 37° C. in a 95% humidified CO₂ incubator with 5% CO₂ atmosphere. The cell lines were passaged twice weekly for maintaining sub-confluent state.

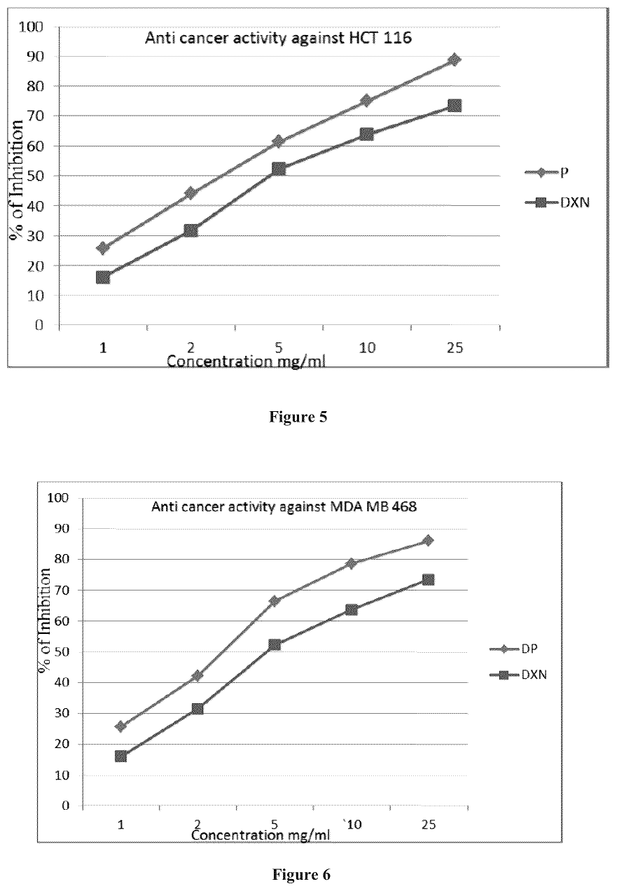

MTT assay was used as cell viability assay. The principle of the assay lies in the fact that the MTT is reduced to formazan crystals by mitochondrial dehydrogenase of the viable cells. The anticancer activity of compounds (3) and (4) was assessed invitro by MTT 3-(4,5-dimethylthiazol-2-yl)-2,5-diphenyltetrazolium bromide assay based on the methods by Mosmann, T and Bose et.al. (Mosmann, T. Journal of Immunological methods. 1983, 65, 55-63; and Bose, D. S.; Idrees, M.; Jakka, N. M.; Rao, J. V. J. Comb. Chem. 2010, 12, 100-110). Doxorubicin (DXN) a known anticancer drug was used as a reference compound in the assay. The treated and untreated cells were washed with PBS (Phosphate buffer saline) and MTT (100 μg/ml) was added and incubated at 37° C. for 5 hr. The MTT was then removed, and the formazan crystals were dissolved by adding DMSO. The absorbance at 540 nm represents the viable cells. The absorbance was recorded using Multiskan spectrum, Thermo scientific. The percentage of cell death was measured for each compound at various concentrations along with IC₅₀ (half maximal inhibitory concentration) values are represent below in Tables 1 and 2. The absorbance from the untreated cells was defined as 100% viable cells. The % viable cells were plotted (Y-axis) against concentration (X-axis). The IC₅₀ values were interpolated from the graphs of FIGS. 5 and 6.

TABLE 1
Activity of Compound (3) denoted as “P” against Human colon carcinoma (HCT-116) cell line

| | Percentage of cell death at various concentrations (μg/mL) | | | | | |
|---|---|---|---|---|---|---|
| Compound | 1.0 | 2.0 | 5.0 | 10.0 | 25.0 | IC₅₀(μg/mL) |
| P | 25.81 ± 0.015 | 44.01 ± 0.028 | 61.45 ± 0.006 | 75.01 ± 0.076 | 88.64 ± 0.018 | 3.8 |
| DXN | 16.12 ± 0.001 | 31.68 ± 0.012 | 52.34 ± 0.015 | 63.76 ± 0.0031 | 73.45 ± 0.060 | 5.0 |

TABLE 2
Activity of Compound (4) denoted as “DP” against
Human breast adenocarcinoma (MDA-MB-468) cell line:

| | Percentage of cell death at various concentrations (μg/mL) | | | | | |
|---|---|---|---|---|---|---|
| Entry | 1.0 | 2.0 | 5.0 | 10.0 | 25.0 | IC₅₀(μg/mL) |
| DP | 25.71 ± 0.003 | 42.27 ± 0.026 | 66.49 ± 0.007 | 78.66 ± 0.018 | 86.10 ± 0.015 | 3.2 |
| DXN | 16.12 ± 0.001 | 31.68 ± 0.012 | 52.34 ± 0.015 | 63.76 ± 0.0031 | 73.45 ± 0.060 | 5.0 |

As can be seen from Tables 1 and 2 and FIGS. 5 and 6, the IC50 values of the exemplary compounds 3 and 4 of the present disclosure are significantly lower than the known compound doxorubicin a standard drug used in the treatment of cancer.

These results illustrate the compound 3 and 4 to possess stronger anticancer activity as compared to known drug doxorubicin.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention and should not be construed so as to limit the scope of the invention or the appended claims in any way.

I claim:

1. A compound of formula (I) or a stereoisomer, a tautomer, a pharmaceutically acceptable salt or a pharmaceutically acceptable solvate thereof, 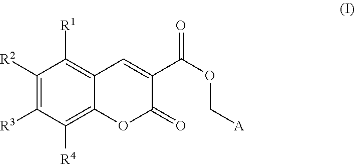
wherein:
R¹ and R² or R² and R³ or R³ and R⁴ combine together with the atoms to which they are attached form a (C₄₋₁₀) heterocyclyl or (C₆₋₁₀) aryl;
R¹ to R⁴ are independently selected from the group consisting of unsubstituted or substituted (C₁₋₆)alkyl, unsubstituted or substituted (C₁₋₆)alkoxy, unsubstituted or substituted (C₃₋₆)cycloalkyl, halogen, and hydroxy; and
A represents ring A or ring A¹ as represented below,

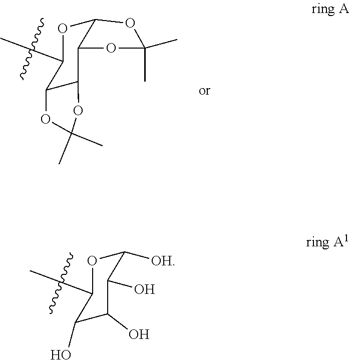 [SMILES: *C1OC2OC(C)(C)OC2C2OC(C)(C)OC12 ; *C1OC(O)C(O)C(O)C1O]

2. The compound of formula (I) as claimed in claim 1 represented by the compound (3) or (4):

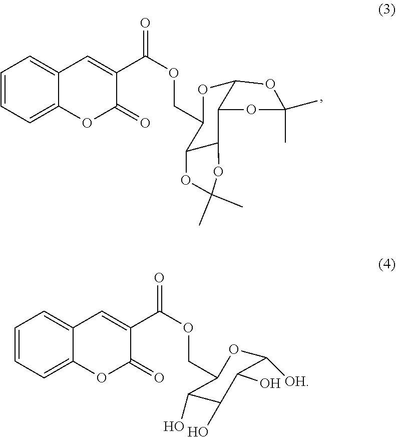 [SMILES: CC1(C)OC2OC(COC(=O)c3cc4ccccc4oc3=O)C3OC(C)(C)OC3C2O1 ; O=C(OCC1OC(O)C(O)C(O)C1O)c1cc2ccccc2oc1=O]

3. A process for preparing a compound of formula (I) or a pharmaceutically acceptable salt thereof, comprising the steps of:
(a) reacting a compound of formula (I) with a compound (2) in presence of coupling agents and polar solvent(s) at a temperature in the range of 25° C. to 30° C. for a time period in the range of 6 hours to 48 hours to obtain a compound of formula (I) (with ring A); 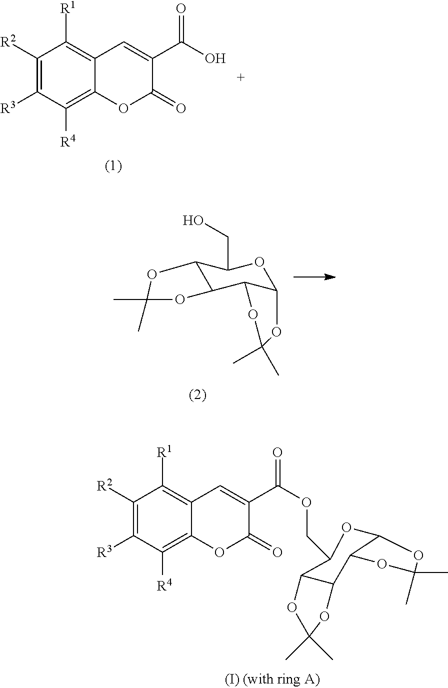
(b) reacting the compound of formula (I) (with ring A) obtained in step (a) with deprotecting agents in polar solvent(s) at a temperature in the range of 25° C. to 30° C. for a time period of 10 to 60 minutes to obtain a compound of forn [symbol] R¹ I; and 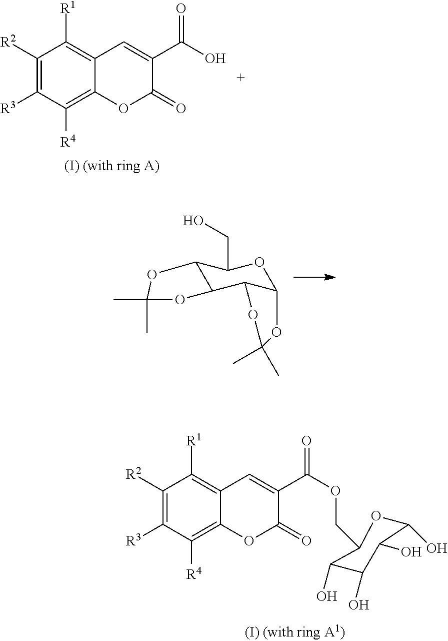
(C) optionally, converting the compound of formula (I) into its salt.

4. The process as claimed in claim 3, wherein, the coupling agent(s) is selected from the group consisting of hexafluorophosphate azabenzotriazole tetramethyl uronium (HATU), tetrafluoroborate azabenzotriazole tetramethyl uronium (TATU), 2-(1H-Benzotriazole-1-yl)-1, 1,3,3-tetramethylaminium tetrafluoroborate (TBTU), hexafluorophosphate benzotriazole tetramethyl uranium (HBTU), and (1-Cyano-2-ethoxy-2-oxo ethylidenaminooxy)dimethylamino-morpholino-carbenium hexafluorophosphate (COMU).

5. The process as claimed in claim 3 wherein, the base is an organic base selected from the group consisting of N-methyl morpholine (NMM), imidazole, urea, carbamate, pyridine, N,N-diisopropylamine and triethylamine.

6. The process as claimed in claim 3 wherein, the polar solvent is from water, dichloromethane, tetrahydrofuran, ethyl acetate, dimethylformamide, acetonitrile, dimethyl sulfoxide, and alcohol.

7. The process as claimed in claim 3 wherein, the deprotecting agent(s) is selected from trifluoroacetic acid, aqueous tert-butyl hydroperoxide (70%), para toluene sulphonic acid, zirconium chloride, and indium trichloride.

8. A process for preparing a compound (3) or compound (4), the process comprising the step of:
(a) reacting compound (1a) with compound (2) in presence of coupling agent(s), a base and polar solvent(s) at a temperature in the range of 25° C. to 30° C. for a time period of 10 to 30 minutes to obtain the compound (3); and 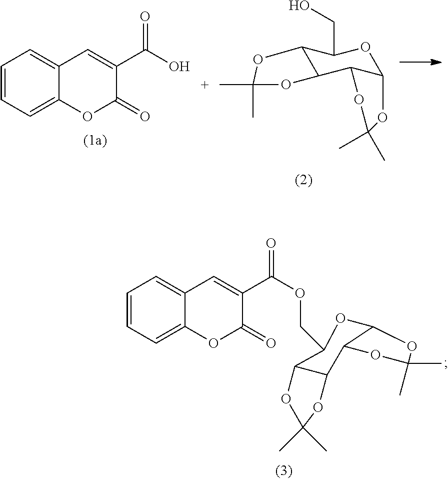
optionally
(b) deprotecting the compound (3) using deprotecting agent(s) and polar solvent(s) at a temperature in the range of 25° C. to 30° C. for a time period of 10 to 60 minutes to obtain a compound (4):

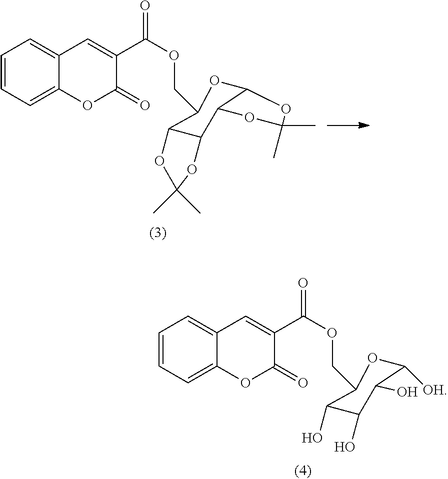 [SMILES: CC1(C)OC2OC(COC(=O)c3cc4ccccc4oc3=O)C3OC(C)(C)OC3C2O1 ; O=C(OCC1OC(O)C(O)C(O)C1O)c1cc2ccccc2oc1=O]

9. The process as claimed in claim 8, wherein, the coupling agent(s) is selected from the group consisting of hexafluorophosphate azabenzotriazole tetramethyl uronium (HATU), tetrafluoroborate azabenzotriazole tetramethyl uronium (TATU), 2-(1H-Benzotriazole-1-yl)-1,1,3,3-tetramethylaminium tetrafluoroborate (TBTU), hexafluorophosphate benzotriazole tetramethyl uranium (HBTU), and (1-Cyano-2-ethoxy-2-oxo ethylidenaminooxy)dimethylamino-morpholino-carbenium hexafluorophosphate (COMU).

10. The process as claimed in claim 8 wherein, the base is an organic base selected from the group consisting of N-methyl morpholine (NMM), imidazole, urea, carbamate, pyridine, N,N-diisopropylamine and triethylamine.

11. The process as claimed in claim 8 wherein, the polar solvent is from water, dichloromethane, tetrahydrofuran, ethyl acetate, dimethylformamide, acetonitrile, dimethyl sulfoxide, and alcohol.

12. The process as claimed in claim 8 wherein, the deprotecting agent(s) is selected from trifluoroacetic acid, aqueous tert-butyl hydroperoxide (70%), para toluene sulphonic acid, zirconium chloride, and indium trichloride.

13. A pharmaceutical composition comprising a therapeutically effective amount of a compound of formula (I) or its pharmaceutically acceptable salt and pharmaceutically acceptable excipients.

14. A method of treating a cancer disease comprising administering a therapeutically effective amount of the compound of formula (I) or a pharmaceutically acceptable salt thereof to a subject in need thereof.
